(19) United States
(12) Patent Application Publication
(10) Pub. No.: US 2007/0059381 A1
(43) Pub. Date: Mar. 15, 2007

(54) Treatment of amd with combination of ingredients

(76) Inventors: Ronnie C. Barker, ARLINGTON, TX (US); John C. Lang, Cedar Hill, TX (US); Ralph P. Stone, Fort Worth, TX (US)

Correspondence Address: Alcon Research;Theresa J. Schultz, R&D Counsel (Q-148), 6201 South Freeway, Fort Worth, TX, 76134-2099, US

(21) Appl. No.: 10/559,443

(22) PCT Filed: Jun. 18, 2004
(86) PCT No.: PCT/US04/19577

§ 371 Date: Dec. 2, 2005

Related U.S. Application Data
(60) Provisional application No. 60/480,054, filed on Jun. 20, 2003.

(51) Int. Cl. A61K 33/32; A61K 31/355; A61K 31/375; A61K 31/015

(52) U.S. Cl. 424/641; 514/458; 514/474; 514/763

(57) ABSTRACT

The present invention is directed to methods for enhancing current treatment regimens for age-related macular degeneration (AMD). The methods of the invention include intraocular delivery of AMD compositions along with antioxidant compositions. Alternatively, the methods of the invention include a daily regimen of ocular vitamin consumption in conjunction with other treatment methods for AMD.

This application claims priority from U.S. Ser. No. 60/480,054, filed Jun. 20, 2003.

### BACKGROUND OF THE INVENTION

1. Field of the Invention

The present invention relates to the fields of ocular vitamins and macular degeneration. More specifically, the present invention relates to methods of enhancing current treatments for macular degeneration with a daily regimen of ocular vitamin consumption or by injecting a composition of antioxidants into the eye.

2. Description of the Related Art

Age-related macular degeneration (AMD) is the leading cause of irreversible loss of vision in people over the age of 65. With onset of AMD there is gradual loss of the light-sensitive photoreceptor cells in the back of the eye, the underlying pigment epithelial cells that support them metabolically, and the sharp central vision they provide. Age is the major risk factor for the onset of AMD: the likelihood of developing AMD triples after age 55. Smoking, light iris color, gender (women are at greater risk), obesity, and repeated exposure to UV radiation also increase the risk of AMD.

Nutraceuticals are a growing aspect of the pharmaceutical market. For diseases such as AMD (exudative type), intervention with supplemental antioxidants and zinc has been shown to slow the progression of some stages of the disease. The Age-Related Eye Disease Study (AREDS, NIH) showed a demonstrable clinical benefit for advanced stages of AMD with daily oral supplementation of antioxidants and other ingredients. The formula used in the study, currently termed the “AREDS formula,” generally includes vitamins A, C, and E, and the minerals zinc and copper. Current thought is that supplementation with compounds such as zeaxanthin and/or lutein exerts a neuroprotective effect by restoring blue light photo protection to the retina, and by reducing the photo-oxidative damage from free radicals generated in photoreception, especially at high luminance.

Products such as Ocuvite® PreserVision™ and ICaps®, touting the findings of the AREDS study, offer formulations including vitamins A, C, and E, and the minerals zinc and copper. Other ocular vitamin formulations may include lutein and zeaxanthin, vitamin B2, folate, vitamin B12, selenium, and manganese or botanically derived antioxidants such as camosic acid and carnosol found in rosemary, other polyphenolics and polyphenolic bioflavonoids typically found in fruits and vegetables, or essential fatty acids like DHA (docosahexaenoic acid) that comprise photoreceptor membranes and possess antioxidant properties in addition to the AREDS ingredients, or some combination thereof. The premise is that oral supplementation will restore normal physiological levels of these compounds which are known to be depleted in the diseased or elderly eye, thereby exerting or regenerating a neuroprotective effect in critical ocular areas (the retina, and especially the macula). Unfortunately, oral administration is a slow means, by itself, for overcoming depleted levels of ocular nutrients, highly dependent on systemic transport and transmembrane migration.

More reliable and uniform benefits of antioxidants from ocular vitamins and/or of other treatments for AMD might be achieved by alternate means of supplementation.

### SUMMARY OF THE INVENTION

The present invention overcomes these and other drawbacks of the prior art by providing a method for treating macular degeneration by administering a therapeutically effective amount of anecortave acetate in conjunction with a daily, or continuously administered, regimen of ocular vitamins. In preferred aspects of the invention, the anecortave acetate is administered via an intraocular cannula, such as that described in U.S. Pat. No. 6,413,245, or is deposited in a depot inserted into the eye, such as those described in U.S. Pat. Nos. 6,416,777; 6,413,540.

The present invention further provides a method for treating macular degeneration by administering a therapeutically effective amount of anecortave acetate and a therapeutically effective amount of an antioxidant composition containing at least one antioxidant. Generally, both compositions will be administered by intraocular cannula, generating a “natural” depot for both drug and antioxidants (accompanied by any necessary methods for controlling dissolution or bioavailability.) The two compositions can be administered at the same time, or the anecortave acetate can be injected through the cannula and then the antioxidant composition injected through the cannula after the anecortave acetate has been placed in the patient's eye. Alternatively, the antioxidant composition may be injected into the eye just prior to the injection of the anecortave acetate. It is further contemplated that the anecortave acetate composition and the antioxidant composition may be placed into a depot, such as that described in U.S. Pat. Nos. 6,416,777; 6,413,540.

In preferred embodiments, the antioxidant composition includes vitamin C, vitamin E, β-carotene, and zinc and copper oxides. In particularly preferred embodiments, the antioxidant composition contains the AREDS formula for ocular vitamins. For example, the antioxidant ratios by weight may contain approximately 48.054% vitamin C, approximately 42.526% vitamin E, approximately 1.850% β-carotene, approximately 7.400% zinc (typically as zinc oxide) and approximately 0.170% copper (as cupric oxide), such as that provided by, but not limited to, the AREDS formula. In other preferred embodiments, the antioxidant composition may contain any one, all or none of the above ingredients, as well as one or more of the following ingredients: manganese, chromium, lutein, zeaxanthin (as in ICaps® L&Z), folate, rosemary or its antioxidants, or other botanically derived antioxidants including polyphenolic bioflavonoids, DHA or other essential fatty acids, or additional B vitamins.

The present invention also includes delivery methods to control the rate of egress from the implanted depot or reservoir, assuring that the local concentrations remain non-toxic and that the duration of delivery of the antioxidants is correlated with the duration of the implanted anecortave acetate.

In another aspect, the method of the present invention includes a daily regimen of ocular vitamin consumption in conjunction with a pharmaceutical or surgical treatment. The pharmaceutical or surgical treatment may be any such treatment described in the art, such as, but not limited to, those described herein.

### DETAILED DESCRIPTION PREFERRED EMBODIMENTS

There are numerous currently described methods for treating AMD. Such methods include administering kinase inhibitors (e.g., U.S. Pat. Nos. 6,559,173; 6,548,503); administering VEGF inhibitors (e.g., U.S. Pat. Nos. 6,114,320; 6,426,335; 6,448,277); administering metalloprotease inhibitors (U.S. Pat. Nos. 6,569,855; 6,566,381); administering an integrin antagonist (e.g., U.S. Pat. Nos. 6,531,494; 6,486,174); administration of anecortave acetate (U.S. Pat. Nos. 6,297,228; 5,770,592; 5,679,666); photodynamic therapy (Asrani & Zeimer, Br J Ophthalmol, 79(8):776-770, August, 1995; Asrani et al, Invest Ophthalmol. Vis Sci, 38(13); 2702-2710, December, 1997; Husain et al, Ophthalmology, 104(8):242-1250, August, 1997; Lin et al, Curr Eye Res, 13(7):513-522, July, 1994); and transpupillary thermotherapy for treating wet AMI (Archives of Ophthalmology and Acta Ophthalmologica Scandinavica) to name just a few.

The present invention provides a means of combining other, known therapies for treatment of macular degeneration with administration of antioxidants, either through the a daily regimen of ocular vitamins in conjunction with another therapy, or through the topical delivery of the antioxidants, through ocular injection (intravitreal, subtenons, subconjunctival, periocular, retrobulbar, juxtascleral), or through intraocular slow release devices or intraocular implants. The therapies listed above, as well as other described methods for treating macular degeneration, may be used in the combination therapy described herein.

The AREDS study, a ten-year, eleven-center, double-masked clinical trial in about 4700 patients with AMD at various stages, found that in the high risk groups, Categories 3 and 4, for developing advanced AMD, there was about a 28% reduction in progression to advanced AMD (as measured by treatment for neovascularization, hemorrhage, central geographic atrophy, etc.) and about 21% reduction in loss of vision (defined as a 15-letter decrease in vision) with supplementation (oral daily 500 [452 minimum] mg vitamin C, 400 IU vitamin E, 15 [17.4 actual minimum] mg beta-carotene, 80 [69.6 actual minimum] mg zinc oxide and 2 [1.6 actual minimum] mg cupric oxide daily). The present invention provides a means of enhancing the effects of current treatments for AMD by combining current, invasive methods of treating the disease with ocular vitamin supplementation. Alternatively, the present invention provides a method for treating AMD by co-administering an antioxidant “cocktail” with current treatments. The antioxidants may be administered prior to administering the pharmaceutical composition to the eye, simultaneously with the pharmaceutical composition, or the antioxidants may be administered in sustained release microencapsulated beadlets.

As used herein, the term “pharmaceutical composition” refers to a composition containing a therapeutically effective amount of a compound in a pharmaceutical vehicle, suitable for administration directly to the eye of the AMD patient by ocular injection with cannulas specific for the type of injection (intravitreal, subtenons, subconjunctival, periocular, retrobulbar, juxtascleral), or through intraocular slow release devices or intraocular or periocular implants. In preferred aspects of the invention, the compound used in the pharmaceutical composition may be anecortave acetate.

Dosage of each constituent of the antioxidant “cocktail” should achieve or approximate the normal physiological level found for each component in the normal eye, generally in the area of the retina or macula. Microencapsulation of antioxidants might provide a better sustained release and prevent cellular toxicity at the site of injection.

In other embodiments, the present invention provides a means of enhancing the effectiveness of treatment of AMD with pharmaceutical preparations, by supplementing such treatment with a daily regimen of ocular vitamins. According to this embodiment, the AMD patient may begin a daily regimen of ocular vitamin supplementation sometime prior to receiving the treatment with the pharmaceutical preparation and continue the daily use of the vitamin indefinitely through the course of the treatment. Prior use is especially recommended for processes (like PDT) that have a high probability of generating an excess of free radicals. Alternatively, the patient may begin the daily regimen of ocular vitamin supplementation at the same time, or on the same day, as beginning the treatment with the pharmaceutical preparation. It is believed, however, that the daily regimen of ocular vitamin supplementation would be effective if begun at any time after the patient begins treatment with the pharmaceutical preparation and continuing indefinitely through the course of treatment.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and structurally related may be substituted for the agents described herein to achieve similar results. All such substitutions and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

United States Patents

5,679,666

5,770,592

6,114,320

6,297,228

6,413,245

6,413,540

6,416,777

6,426,335

6,448,277

6,486,174

6,531,494

6,548,503

6,559,173

6,566,381

6,569,855

Other Publications
Asrani & Zeimer, Br. J. Ophthalm. 79(8):776-770 (1995).
Asrani et al., Invest. Ophthalmol. Vis. Sci. 38(13):2702-2710 (1997).
Husain et al., Ophthalmology 104(8):1242-1250 (1997).
Lin et al., Curr. Eye Res. 13(7):513-522 (1994).

We claim:

1. A method for treating macular degeneration, said method comprising administering a therapeutically effective amount of anecortave acetate in conjunction with a daily regimen of ocular vitamins.

2. The method of claim 1, wherein said anecortave acetate is administered via an intraocular cannula.

3. A method for treating macular degeneration, said method comprising administering a therapeutically effective amount of anecortave acetate and a therapeutically effective amount of an antioxidant composition comprising at least one antioxidant, wherein said administering is by intraocular cannula.

4. The method of claim 3, wherein either the anecortave acetate, the antioxidant composition, or both are administered by means of an implant capable of sustaining the delivery, wherein the implant is ocular, periocular, juxtascleral or the like.

5. The method of claim 3, wherein the antioxidant composition comprises vitamin C, vitamin E, β-carotene, and zinc oxide.

6. The method of claim 3, wherein said administering is simultaneous.

7. The method of claim 3, wherein said anecortave acetate is administered first and the antioxidant composition is administered second.

8. The method of claim 5, wherein the antioxidant composition comprises a blend consisting of a ratio equivalent to the following amounts of antioxidant: about 48% vitamin C, about 42.5% vitamin E, about 1.9% β-carotene, about 7.4% zinc and about 0.2% copper.

9. The method of claim 3, wherein the antioxidant composition comprises a blend of including folate, rosemary or its antioxidants or other botanically derived antioxidants including polyphenolic bioflavonoids, DHA or other essential fatty acids, or additional B vitamins.

10. The method of claim 3, wherein the antioxidant composition comprises zeaxanthin.

11. The method of claim 10, wherein the antioxidant composition further comprises lutein.

12. A method for treating macular degeneration, said method comprising a daily regimen of ocular vitamin consumption in conjunction with a pharmaceutical or surgical treatment.
